(19) United States
(12) Patent Application Publication
(10) Pub. No.: US 2025/0161509 A1
(43) Pub. Date: May 22, 2025

(54) FLUID STERILIZATION DEVICE

(71) Applicant: TOYODA GOSEI CO., LTD., Kiyosu-shi (JP)

(72) Inventors: Satoshi WADA, Kiyosu-shi (JP); Takeshi MATSUSHIMA, Kiyosu-shi (JP); Kazushige SUZUKI, Kiyosu-shi (JP)

(21) Appl. No.: 18/945,056

(22) Filed: Nov. 12, 2024

(30) Foreign Application Priority Data
Nov. 16, 2023 (JP) 2023-194891

(51) Int. Cl. A61L 2/10; A61L 2/24; A61L 2/26; A61L 9/20; C02F 1/32

(52) U.S. Cl. CPC A61L 2/10; A61L 2/24; A61L 2/26; A61L 9/20; C02F 1/325; A61L 2202/11; A61L 2202/122; A61L 2202/14; A61L 2209/111; A61L 2209/12; C02F 2201/3222; C02F 2201/3227; C02F 2201/326; C02F 2303/04

(57) ABSTRACT

A fluid sterilization device includes: a flow path tube forming a flow path space through which a fluid flows and having a side wall, a first end, and a second end, the side wall provided with an inlet at a side of the first end and an outlet at a side of the second end; and a light source unit disposed at a position closer to the inlet in the flow path space and configured to emit ultraviolet light to the second end. The light source unit includes a light emitting element, a support portion provided to protrude from an end surface of the first end toward the second end, provided at a position corresponding to the inlet, and formed to be tapered toward the second end, and an accommodation portion that is provided at a front end of the support portion and accommodates the light emitting element.

### CROSS-REFERENCE TO RELATED APPLICATIONS

This application is based on and claims priority under 35 USC 119 from Japanese Patent Application No. 2023-194891 filed on Nov. 16, 2023, the entire content of which is incorporated herein by reference.

### TECHNICAL FIELD

The present disclosure relates to a fluid sterilization device.

### BACKGROUND ART

A sterilization device that sterilizes bacteria and viruses in running water by irradiating the water with ultraviolet light is known. A mercury lamp is widely used as a light source. Since the mercury lamp uses mercury and thus the mercury lamp is highly toxic, there is a problem that the mercury lamp has a large environmental load. Further, there is also a problem that use of a mercury lamp increases a size of the sterilization device. Therefore, the mercury lamp has been replaced with an ultraviolet LED.

In a water sterilization device using an ultraviolet LED, the ultraviolet LED generates a large amount of heat, and it is necessary to efficiently cool the ultraviolet LED. In the related art, heat radiation has been performed by providing a heat sink, but the device is increased in size and weight.

JP2022-173327A and JP2019-18198A disclose a fluid sterilization device that uses an ultraviolet LED for cooling water to be sterilized without using a heat sink. JP2022-173327A and JP2019-18198A disclose a configuration in which a columnar light source unit that protrudes from one end toward the other end of a flow path tube is provided.

JP2022-173327A and JP2019-18198A describe that the light source unit is cooled by directing water from an inlet to a base of the columnar portion.

### SUMMARY OF INVENTION

However, in structures in JP2022-173327A and JP2019-18198A, the light source unit is not sufficiently cooled, and cooling efficiency needs to be further improved.

Aspects of the present disclosure relate to providing a fluid sterilization device having high cooling efficiency for a light source unit.

According to an aspect of the present disclosure, there is provided a fluid sterilization device including:
a flow path tube forming a flow path space through which a fluid flows and having a side wall, a first end, and a second end, the side wall being provided with an inlet formed at a side of the first end and an outlet formed at a side of the second end; and
a light source unit disposed at a position closer to the inlet in the flow path space and configured to emit ultraviolet light to the second end, in which
the light source unit includes
   a light emitting element that emits ultraviolet light,
   a support portion provided to protrude from an end surface of the first end of the flow path tube toward the second end, provided at a position corresponding to the inlet, and formed to be tapered toward the second end, and
   an accommodation portion that is provided at a front end of the support portion and accommodates the light emitting element.

According to the above aspect of the present disclosure, the support portion of the light source unit is formed to be tapered toward the second end, and a flow path is formed such that a fluid that flows in from the inlet of the flow path tube flows toward the accommodation portion of the light source unit. As a result, the light source unit may be efficiently cooled by the fluid. Further, a flow path may be formed through which the fluid that flows in from the inlet of the flow path tube flows toward the outlet in the second end by the support portion.

### BRIEF DESCRIPTION OF DRAWINGS

Exemplary embodiment(s) of the present invention will be described in detail based on the following figures, wherein:

FIG. 1 is a view schematically illustrating a configuration of a fluid sterilization device according to Embodiment 1;

FIG. 2A is a view illustrating a cross section of a flow path tube at an inlet, the cross section being in a direction perpendicular to a central axis of the flow path tube;

FIG. 2B is a view illustrating a cross section of the flow path tube at an outlet, the cross section being in a direction perpendicular to the central axis of the flow path tube;

FIG. 3 is a view schematically illustrating a flow of a fluid near the inlet;

FIG. 4 is a view illustrating a simulation result of the flow of the fluid;

FIGS. 5A to 5C are views schematically illustrating configurations of a light source unit of a fluid sterilization device according to Embodiment 2;

FIG. 6 is a view schematically illustrating a configuration of a light source unit of a fluid sterilization device according to Embodiment 3;

FIG. 7 is a view schematically illustrating a configuration of a fluid sterilization device according to Modification 1 of Embodiment 1; and

FIG. 8 is a view schematically illustrating a configuration of a fluid sterilization device according to Modification 2 of Embodiment 1.

### DESCRIPTION OF EMBODIMENTS

A fluid sterilization device includes: a flow path tube forming a flow path space through which a fluid flows and having a side wall, a first end, and a second end, the side wall the side wall being provided with an inlet formed at a side of the first end and an outlet formed at a side of the second end, and a light source unit disposed at a position closer to the inlet in the flow path space and configured to emit ultraviolet light to the second end. The light source unit includes a light emitting element that emits ultraviolet light, a support portion provided to protrude from an end surface of the first end of the flow path tube toward the second end, provided at a position corresponding to the inlet, and formed to be tapered toward the second end, and an accommodation portion that is provided at a front end of the support portion and accommodates the light emitting element.

In the fluid sterilization device, the support portion may have a truncated cone shape. A helical flow is easily formed.

In the fluid sterilization device, the accommodation portion may be formed to extend from the front end of the support portion toward an outside of the support portion in a radial direction over an entire periphery of the front end of the support portion. The fluid may be brought into contact with a back surface of the accommodation portion, and the accommodation portion may be efficiently cooled. In this case, a surface of the accommodation portion at side of the support portion may be provided with a helical groove or a protrusion. Further, a surface of the accommodation portion at side of the support portion may be provided with a radial groove or a protrusion.

In the fluid sterilization device, the light emitting element may be positioned outside the support portion when viewed from a central axis direction of the support portion, in the accommodation portion. The fluid may be brought into contact with a region of the back surface of the accommodation portion directly below the light emitting element, and the accommodation portion may be efficiently cooled.

In the fluid sterilization device, the accommodation portion may include a peripheral wall that is formed on an outer surface of the accommodation portion at a side of the support portion, protrudes toward the support portion, and surrounds at least a portion of the front end of the support portion. The fluid may be retained in a recess surrounded by the peripheral wall, and the accommodation portion may be efficiently cooled.

In the fluid sterilization device, a flow path direction of the inlet may coincide with a direction toward a position offset from a central axis of the flow path tube, the support portion may be provided at a position including the central axis of the flow path tube, and the fluid that flows in from the inlet may circulate around the support portion. A helical flow may be easily formed.

The fluid sterilization device may further include a second light source unit disposed at a position closer to the outlet in the flow path space and configured to emit ultraviolet light to the first end. The outlet may be formed at a side of the second end on the side wall. The second light source unit may include a second light emitting element that emits ultraviolet light, a second support portion provided to protrude from an end surface of the second end of the flow path tube toward the first end, provided at a position corresponding to the outlet, and formed to be tapered toward the first end, and a second accommodation portion that is provided at a front end of the second support portion and accommodates the second light emitting element. The second support portion may have a truncated cone shape, and the second accommodation portion may be formed to extend from the front end of the second support portion toward an outside of the second support portion in a radial direction over an entire periphery of the front end of the second support portion. The fluid may be reflected by the second support portion in the radial direction of the flow path tube, and the second accommodation portion may be efficiently cooled by the fluid.

A fluid sterilization device includes: a flow path tube that forming a flow path space through which a fluid flows and having a side wall, a first end, and a second end, the side wall being provided with an inlet formed at a side of the first end and an outlet formed at a side of the second end; and a second light source unit disposed at a position closer to the outlet in the flow path space and configured to emit ultraviolet light to the first end. The second light source unit includes a second light emitting element that emits ultraviolet light, a second support portion provided to protrude from an end surface of the second end of the flow path tube toward the first end, provided at a position corresponding to the outlet, and formed to be tapered toward the first end, and a second accommodation portion that is provided at a front end of the second support portion and accommodates the light emitting element.

In the fluid sterilization device, a flow path direction of the outlet may coincide with a direction toward a position offset from a central axis of the flow path tube, the second support portion may be provided at a position including the central axis of the flow path tube, and the fluid that circulates around a periphery of the second support portion may flow out from the outlet.

In the fluid sterilization device, flow path directions of the inlet and the outlet may coincide with a direction toward a position offset from a central axis of the flow path tube, the fluid that flows through the flow path space may form a helical flow, and the inlet and the outlet may be formed such that, when the flow path tube is viewed from a central axis direction, inflow directions of the inlet and the outlet coincide with a tangential direction of rotation of the helical flow centered on the central axis of the flow path tube. A helical flow is more easily formed and maintained.

The fluid sterilization device may further include a light intensity sensor that protrudes from an inner wall surface of the flow path tube toward a central axis of the flow path tube. The light intensity sensor may also serve as a flow guide plate, and the helical flow may be easily maintained.

In the fluid sterilization device, the flow path tube may include a helical groove on an inner wall surface of the flow path tube. The helical flow may be easily maintained.

### Embodiment 1

### 1. Overview of Configuration of Fluid Sterilization Device 1

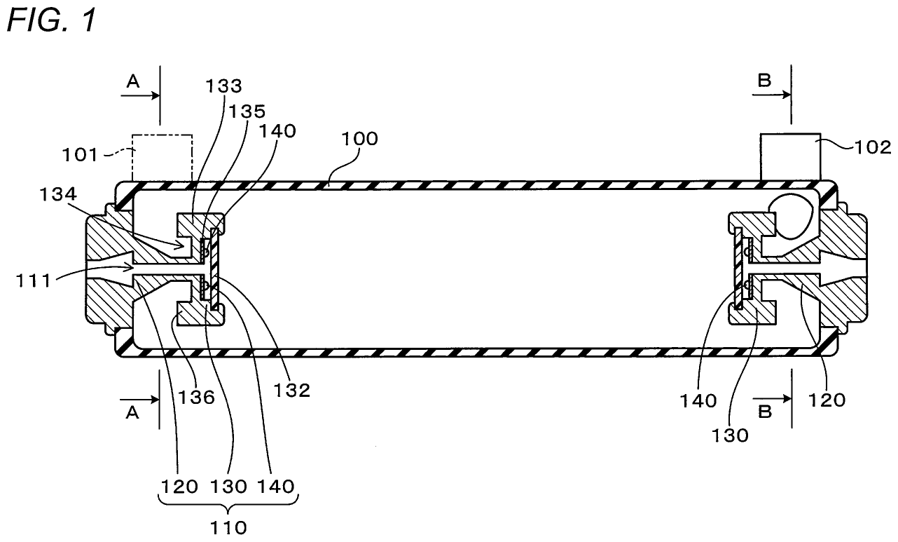

FIG. 1 is a view schematically illustrating a configuration of a fluid sterilization device 1 according to Embodiment 1. As illustrated in FIG. 1, the fluid sterilization device 1 according to Embodiment 1 includes a flow path tube 100 and two light source units 110. Further, the light source unit 110 includes LED packages 140, a support portion 120, and an accommodation portion 130.

The fluid sterilization device 1 according to Embodiment 1 is a device that causes a fluid to flow from an inlet 101 of the flow path tube 100 to a flow path space inside the flow path tube 100, irradiates the fluid with ultraviolet light from the light source unit 110 to sterilize the fluid, and discharges the sterilized fluid from an outlet 102. The fluid to be sterilized may be a gas or a liquid, and may be a mixture of a gas and a liquid, a mixture of a gas and a powdery solid, or the like as long as the fluid has fluidity.

When the fluid is a liquid, examples thereof include water, oil, alcohol, and a solution containing the same as a solvent.

### 2. Details of Each Configuration of Fluid Sterilization Device 1

Next, each configuration of the fluid sterilization device 1 will be described in detail.

### 2-1. Configuration of Flow Path Tube 100

The flow path tube 100 has a cylindrical tube shape and has a cylindrical column-shaped space therein. This space is a flow path space through which the fluid to be sterilized flows. Both ends of the flow path tube 100 are each provided with a light source unit 110. Further, a side wall on one end surface (first end) of the flow path tube 100 in an axial direction is provided with an inlet 101, and a side wall on the other end surface (second end) is provided with an outlet 102.

Examples of a material for the flow path tube 100 include SUS, titanium, and polytetrafluoroethylene (PTFE). An inner wall surface of a resin material resistant to ultraviolet light may be covered with a material having a high reflectance to ultraviolet light. The resin material having resistance to the ultraviolet light is, for example, vinyl chloride. Further, examples of the material having a high reflectance to the ultraviolet light include aluminum and PTFE. Further, an outer wall surface of a material that transmits ultraviolet light may be covered with a material having a high reflectance to ultraviolet light. Examples of the material that transmits the ultraviolet light include sapphire, ultraviolet-transmitting glass, fluororesin, and acrylic resin.

An inner wall surface of the flow path tube 100 preferably has an arithmetic mean roughness Ra of 0.2 nm to 10 μm. A resistance of the inner wall surface decreases, allowing the flow to be easily maintained. The arithmetic mean roughness Ra is more preferably 0.2 nm to 3 μm, and still more preferably 0.2 nm to 1 μm.

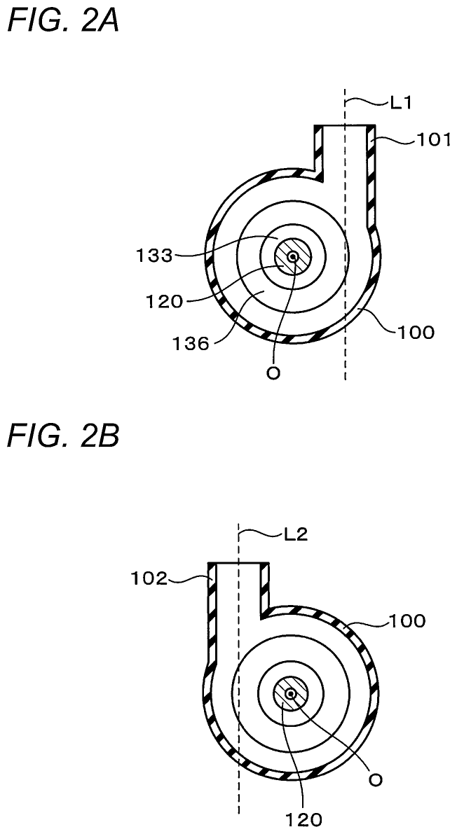

FIGS. 2A and 2B are views illustrating positions of the inlet 101 and the outlet 102. FIG. 2A illustrates a position of the inlet 101 and FIG. 2B illustrates a position of the outlet 102. Further, FIGS. 2A and 2B illustrate cross sections perpendicular to a central axis of the flow path tube 100. FIG. 2A is a cross section taken along line A-A in FIG. 1 and FIG. 2B is a cross section taken along line B-B in FIG. 1, when viewed in a direction from the first end toward the second end thereof.

As illustrated in FIG. 2A, the inlet 101 is disposed such that an inflow direction of the fluid that flows in from the inlet 101 is offset from a center O of the flow path tube 100. That is, a central axis L1 of the inlet 101 is disposed offset so as not to pass through the center O of the flow path tube 100. By offsetting the position of the inlet 101 in this manner, a helical flow can be formed in the flow path space in the flow path tube 100, and a tangential direction of the helical flow is a direction of the central axis L1 of the inlet 101.

As illustrated in FIG. 2B, an outflow direction of the outlet 102 is also disposed offset from the center O of the flow path tube 100. That is, a center axis L2 of the outlet 102 is disposed offset so as not to pass through the center O of the flow path tube 100. Accordingly, the helical flow may be maintained even on an outlet 102 side, and the tangential direction of the helical flow becomes a direction of the central axis L2 of the outlet 102.

### 2-2. Configuration of Light Source Unit 110

The light source unit 110 includes the support portion 120, the LED packages 140, and the accommodation portion 130 that accommodates the LED packages 140. Hereinafter, the light source unit 110 provided at the first end will be described, and the light source unit 110 provided at the second end has the same configuration.

As illustrated in FIG. 1, the support portion 120 protrudes from the first end of the flow path tube 100 toward the second end, and has a truncated cone-shaped portion. A central axis of the support portion 120 coincides with the central axis of the flow path tube 100. An inclination angle of a side surface of the truncated cone (angle to a bottom surface) is, for example, 30 to 70°. One end of the support portion 120 on a side with a larger diameter is connected to the first end of the flow path tube 100, and one end of the support portion 120 on a side with a smaller diameter is connected to the accommodation portion 130.

A shape of the support portion 120 is not limited to a truncated cone shape, and may be any shape that tapers toward the second end of the flow path tube 100. Although the shape may be tapered in a stepwise manner, it is preferable that the shape is continuously tapered. For example, a truncated pyramid shape may be used. However, in order to form a helical flow, a truncated cone shape is preferable. Further, the entire support portion 120 may not be a truncated cone, and one portion may be a truncated cone and the other portion may be a column. For example, as illustrated in FIG. 1, a side of a front end of the support portion 120 connected to the accommodation portion 130 may have a cylindrical column shape, and the other portion may have a truncated cone shape.

The accommodation portion 130 is connected to the front end of the support portion 120. The accommodation portion 130 accommodates the LED packages 140. The accommodation portion 130 includes a glass plate 132, a base portion 133, and a substrate 135. The base portion 133 has a cylindrical column-shaped box shape whose upper surface is opened, and an outer bottom surface is connected to the front end of the support portion 120. The substrate 135 is disposed on a bottom surface inside the box, and the LED packages 140 are mounted on the substrate 135. The glass plate 132 is provided on an upper surface of the box and seals an inside of the box. The glass plate 132 is made of a material that transmits the ultraviolet light from the LED packages 140, such as quartz or sapphire. A photocatalytic film that transmits the ultraviolet light may be provided on a surface of the glass plate 132 to inhibit propagation of bacteria and prevent organic contamination on the glass plate 132. The glass plate 132 is not limited to a flat plate, and may be lenticular. For example, the glass plate 132 may be a TIR lens, a fly-eye lens, and a Fresnel lens.

As illustrated in FIG. 1, the base portion 133 is formed to extend from the front end of the support portion 120 to an outside of the support portion 120 in the radial direction over an entire periphery of the front end of the support portion 120. Therefore, a back surface of the base portion 133 is in contact with the flow path space except for a region connected to the support portion 120.

The base portion 133 includes a peripheral wall 136 that protrudes toward the first end in an outer peripheral region of the back surface thereof, and includes a recess 134 surrounded by the back surface of the base portion 133 and the peripheral wall 136. The peripheral wall 136 does not need to be provided over the entire peripheral, and may be partially provided. If the peripheral wall 136 is provided over the entire peripheral, air may be accumulated in the recess 134 and a cooling efficiency may be reduced. Further, it is preferable that the peripheral wall 136 is provided outside the LED packages 140 when viewed from a central axis direction of the flow path tube 100. That is, it is preferable that the LED packages 140 are positioned in a region of the recess 134. The base portion 133 may be more efficiently cooled.

In Embodiment 1, the base portion 133 has a cylindrical column-shaped box shape, and any shape may be used as long as it has a box shape. For example, the base portion 133 may be a square prism-shaped box shape (square shape). However, from a viewpoint of generating a helical flow, it is preferable to form a cylindrical column-shaped box as that in Embodiment 1.

A material of the support portion 120 and the base portion 133 is preferably a metal material having high thermal conductivity such as SUS or aluminum, or a high heat dissipation resin mixed with a thermally conductive filler. Further, titanium may also be used and a surface thereof oxidized to form a photocatalytic film. The propagation of bacteria on the support portion 120 and the base portion 133 may be inhibited.

The LED packages 140 are mounted on the substrate 135. A plurality of LED packages 140 may be mounted. Two LED packages 140 are mounted in FIG. 1. The LED package 140 includes an LED, a substrate on which the LED is mounted, and a lens that seals the LED.

The LED is a light emitting element that emits ultraviolet light. A wavelength of the ultraviolet light is preferably 250 to 285 nm, which is a wavelength having high sterilization efficiency. A plurality of LEDs may be provided in one LED package 140.

It is preferable that the LED package 140 is mounted in a region outside the support portion 120 when viewed from the central axis direction of the flow path tube 100. Since the fluid may be brought into contact with a region of the back surface of the base portion 133 directly below the LED package 140, the accommodation portion 130 may be efficiently cooled.

In Embodiment 1, the packaged LED package 140 is mounted on the substrate 135. The LED may be directly mounted on the substrate 135.

A through continuous hole 111 is provided at a center of the support portion 120 and the accommodation portion 130. The hole 111 is a hole through which a wiring cable that supplies power to the LED package 140 and circuit components on a mounting substrate is inserted. The wiring cable is drawn into the mounting substrate through the hole.

### 3. Regarding Flow Path of Fluid

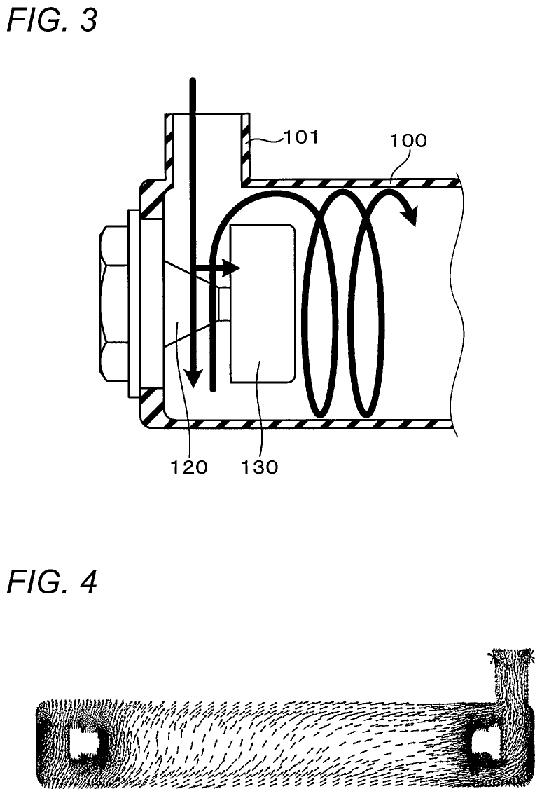

Next, a flow path of the fluid in the flow path space will be described. FIG. 3 is a view schematically illustrating the flow path of the fluid in a vicinity of the first end of the flow path tube 100. As illustrated in FIG. 3, the fluid that flows from the first inlet 101 into the flow path space in the flow path tube 100 hits a side surface of the truncated cone-shaped portion of the support portion 120 and is reflected in an axial direction of the support portion 120 because the side surface is inclined, forming a flow path toward the accommodation portion 130. Therefore, the fluid may be efficiently brought into contact with the accommodation portion 130, and the cooling efficiency may be improved.

Further, since the base portion 133 is formed to extend from the front end of the support portion 120 to the outside of the support portion 120 in the radial direction along the entire periphery of the front end of the support portion 120, the fluid may be brought into contact with the back surface of the base portion 133. In particular, the fluid is brought into contact with the region of the back surface of the base portion 133 which is directly below the LED package 140. Therefore, the base portion 133 may be efficiently cooled.

In addition, since the inlet 101 is offset, as illustrated in FIG. 3, a flow path that circulates around the support portion 120 is formed. Further, since the support portion 120 is shaped to be tapered toward the second end, the fluid circulates around the support portion 120 and goes toward the axial direction of the flow path tube 100. Therefore, a helical flow is formed in the flow path space. By forming the helical flow, residence time of the fluid in the flow path space is increased, and irradiation time of the ultraviolet light to the fluid is increased, thereby improving the sterilization efficiency.

In the light source unit 110 on the outlet 102 side, since the support portion 120 has a shape that tapers toward the first end, the fluid can be reflected in the radial direction of the flow path tube 100 by the support portion 120, and the residence time of the fluid can be increased, thereby enabling the accommodation portion 130 to be efficiently cooled.

### 4. Simulation Results

FIG. 4 is a view illustrating a result of calculating a flow velocity vector of a fluid in a flow path space by simulation. As illustrated in FIG. 4, it is understood that the fluid forms a helical flow in the flow path space, and the helical flow is maintained up to the outlet 102. As described above, it can be seen that the helical flow may be generated by offsetting the inlet 101 and the outlet 102 and setting the shape of the support portion 120 to be tapered toward the second end.

### 5. Summary of Effects

As described above, according to the fluid sterilization device in Embodiment 1, since the support portion 120 of the light source unit 110 has a truncated cone shape, the fluid flows toward the accommodation portion 130, and the accommodation portion 130 may be efficiently cooled by the fluid.

Further, according to the fluid sterilization device in Embodiment 1, a helical flow may be easily formed in the flow path space. As a result, the residence time of the fluid in the flow path space may be increased, and the sterilization efficiency may be improved.

Further, since the accommodation portion 130 is formed to extend from the front end of the support portion 120 to the outside of the support portion 120 in the radial direction, the fluid may be brought into contact with the back surface of the accommodation portion 130. Therefore, the accommodation portion 130 may be efficiently cooled.

Further, since the peripheral wall 136 is provided, the fluid may be retained in the recess 134 on the back surface of the accommodation portion 130, and the cooling efficiency may be improved.

Further, by forming the shape of the support portion 120 to be tapered toward the second end, heat of the accommodation portion 130 may be efficiently diffused to the support portion 120, and the support portion 120 may be efficiently cooled by the fluid.

### Embodiment 2

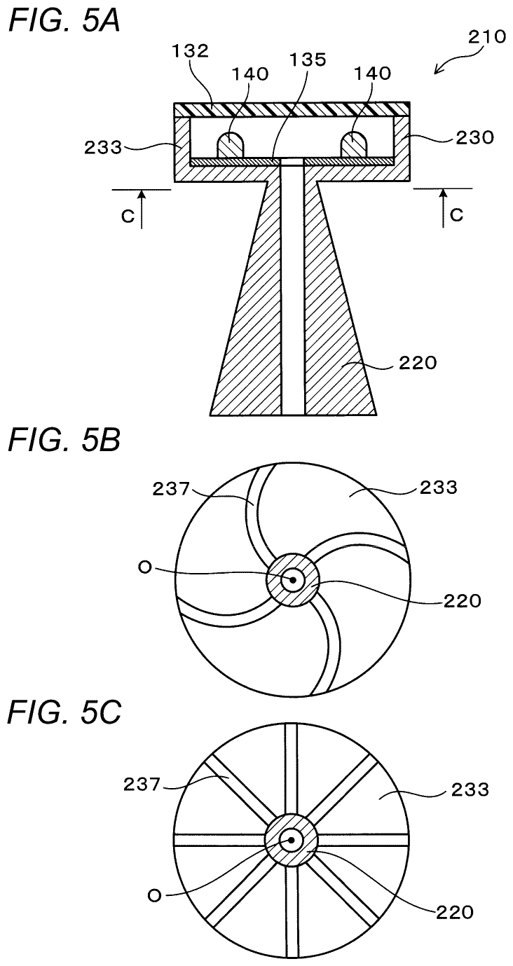

FIGS. 5A to 5C are views schematically illustrating configurations of a light source unit 210 of a fluid sterilization device according to Embodiment 2. As illustrated in FIG. 5A, the light source unit 210 includes a support portion 220 and an accommodation portion 230. The support portion 220 has the same configuration as the support portion 120 according to Embodiment 1. The accommodation portion 230 has a configuration in which the base portion 133 of the accommodation portion 130 according to Embodiment 1 is replaced with a base portion 233, and other configurations are the same as those of the accommodation portion 130. The base portion 233 has a configuration in which the peripheral wall 136 is eliminated from the base portion 133, and an outer peripheral region on a back surface of the base portion 233 is flat.

In Embodiment 2, the effect of retaining the fluid on the back surface of the accommodation portion 230 is not obtained by the peripheral wall 136, whereas other effects can be obtained in the same manner as in Embodiment 1.

In Embodiment 2, in the light source unit 210 on an inlet 101 side, a groove 237 may be provided on the back surface of the accommodation portion 230 (back surface of the base portion 233), and the fluid may be guided from a center side to an outer periphery side of the back surface of the accommodation portion. Alternatively, a wall-shaped protrusion may be provided instead of the groove 237.

FIGS. 5B and 5C are each a sectional view illustrating a cross section taken along line C-C in FIG. 5A. FIG. 5B illustrates a case in which a helical groove 237 is provided on the back surface of the base portion 233. A center of the helical is a center of the support portion 220. By providing such helical groove 237, contact time between the fluid and the accommodation portion 230 is increased, so that the accommodation portion 230 may be efficiently cooled. Further, a flow path swirling toward the outer periphery of the back surface of the accommodation portion 230 may be formed, and the fluid passing between an inner wall of the flow path tube 100 and the accommodation portion 230 easily forms a helical flow.

FIG. 5C illustrates a case in which a radial groove 237 is provided on the back surface of the base portion 233. By providing such groove 237, the fluid may be guided to the outer periphery.

The light source unit 210 on the outlet 102 side may also be provided with the groove 237 as illustrated in FIGS. 5B and 5C. The accommodation portion 230 may be efficiently cooled. Further, the fluid that passes between the inner wall of the flow path tube 100 and the accommodation portion 230 may be guided to the support portion 220, and then a smooth flow path toward the outlet 102 may be formed through reflection by the support portion 220.

### Embodiment 3

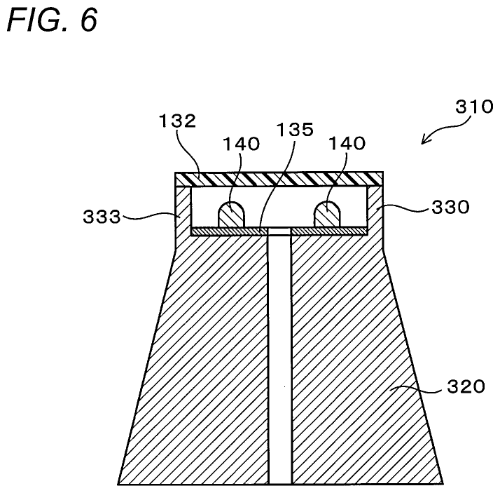

FIG. 6 is a view schematically illustrating a configuration of a light source unit 310 of a fluid sterilization device according to Embodiment 3. As illustrated in FIG. 6, the light source unit 310 includes a support portion 320 and an accommodation portion 330. The support portion 320 has the same configuration as the support portion 120 according to Embodiment 1. A back surface of a base portion 333 of the accommodation portion 330 coincides with a front end of the support portion 320. There is no portion that protrudes outside the support portion 320 in the radial direction. The other configurations are the same as those of the accommodation portion 130.

In Embodiment 3, the fluid cannot be brought into contact with the back surface of the accommodation portion 130 for cooling, whereas other effects can be obtained in the same manner as in Embodiment 1.

### Modification 1 of Embodiment 1

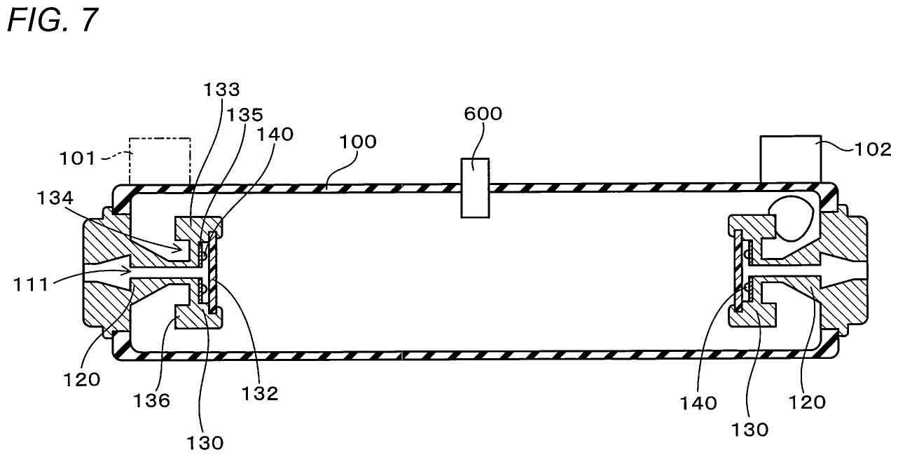

FIG. 7 is a view schematically illustrating a configuration of a fluid sterilization device according to Modification 1 of Embodiment 1. As illustrated in FIG. 7, the fluid sterilization device according to Modification 1 includes a light intensity sensor 600 in a central portion of the flow path tube 100. Other configurations are the same as those of the fluid sterilization device according to Embodiment 1.

The light intensity sensor 600 is a sensor that detects an intensity of the ultraviolet light in a central portion in the flow path tube 100. For example, outputs of the two light source unit 110 are controlled such that the intensity of the ultraviolet light in the central portion is equal to or greater than a predetermined value or more.

Further, the light intensity sensor 600 also serves as a flow guide plate. The light intensity sensor 600 is a wall-shaped protrusion that is provided on the inner wall of the flow path tube 100 and protrudes toward the central axis of the flow path tube 100. The light intensity sensor 600 has a wall shape along a direction of the helical flow, thereby allowing the helical flow to be maintained in the central portion of the flow path tube 100.

### Modification 2 of Embodiment 1

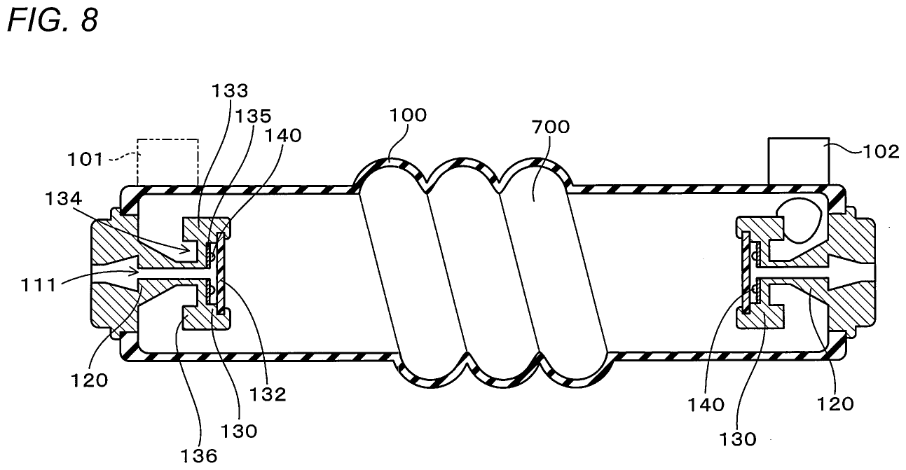

FIG. 8 is a view schematically illustrating a configuration of a fluid sterilization device according to Modification 2 of Embodiment 1. As illustrated in FIG. 8, the fluid sterilization device according to Modification 2 has a configuration in which a helical groove 700 is provided in the inner wall of the flow path tube 100, and the other configurations are the same as those of the fluid sterilization device according to Embodiment 1. By providing the helical groove 700 in the flow path tube 100, the helical flow may be easily maintained in the flow path space, and the sterilization efficiency may be improved.

### (Other Modifications)

In the fluid sterilization device according to Embodiments 1 to 5, the light source unit 110 is provided on each of the inlet 101 side and the outlet 102 side. The light source unit 110 may be provided only on the inlet 101 side when the flow path tube 100 is short. In this case, by disposing a reflective member that reflects ultraviolet light on the end surface on the second end side and irradiating the fluid with reflected light of the ultraviolet light by the reflective member, the sterilization efficiency may be improved. Further, the light source unit 110 may be provided only on the outlet 102 side. Also in this case, by providing the reflective member on the end surface on the first end side, the sterilization efficiency may be improved. PTFE, SUS, Ti, or the like may be used for the reflective member. Further, a resin such as vinyl chloride coated with fluorine may also be used.

What is claimed is:

1. A fluid sterilization device comprising:
a flow path tube forming a flow path space through which a fluid flows and having a side wall, a first end, and a second end, the side wall being provided with an inlet formed at a side of the first end and an outlet formed at a side of the second end; and
a light source unit disposed at a position closer to the inlet in the flow path space and configured to emit ultraviolet light to the second end, wherein
the light source unit includes
a light emitting element that emits ultraviolet light,
a support portion provided to protrude from an end surface of the first end of the flow path tube toward the second end, provided at a position corresponding to the inlet, and formed to be tapered toward the second end, and
an accommodation portion that is provided at a front end of the support portion and accommodates the light emitting element.

2. The fluid sterilization device according to claim 1, wherein
the support portion has a truncated cone shape.

3. The fluid sterilization device according to claim 1, wherein
the accommodation portion is formed to extend from the front end of the support portion toward an outside of the support portion in a radial direction over an entire periphery of the front end of the support portion.

4. The fluid sterilization device according to claim 3, wherein
the light emitting element is positioned outside the support portion when viewed from a central axis direction of the support portion, in the accommodation portion.

5. The fluid sterilization device according to claim 3, wherein
the accommodation portion includes a peripheral wall that is formed on an outer surface of the accommodation portion at a side of the support portion, protrudes toward the support portion, and surrounds at least a portion of the front end of the support portion.

6. The fluid sterilization device according to claim 1, wherein
a flow path direction of the inlet coincides with a direction toward a position offset from a central axis of the flow path tube,
the support portion is provided at a position including the central axis of the flow path tube, and the fluid that flows in from the inlet circulates around the support portion.

7. The fluid sterilization device according to claim 1, further comprising:
a second light source unit disposed at a position closer to the outlet in the flow path space and configured to emit ultraviolet light to the first end, wherein
the outlet is formed at a side of the second end at the side wall,
the second light source unit includes
a second light emitting element that emits ultraviolet light,
a second support portion provided to protrude from an end surface of the second end of the flow path tube toward the first end, provided at a position corresponding to the outlet, and formed to be tapered toward the first end, and
a second accommodation portion that is provided at a front end of the second support portion and accommodates the second light emitting element,
the second support portion has a truncated cone shape, and
the second accommodation portion is formed to extend from the front end of the second support portion toward an outside of the second support portion in a radial direction over an entire periphery of the front end of the second support portion.

8. A fluid sterilization device comprising:
a flow path tube that forming a flow path space through which a fluid flows and having a side wall, a first end, and a second end, the side wall being provided with an inlet formed at a side of the first end and an outlet formed at a side of the second end; and
a second light source unit disposed at a position closer to the outlet in the flow path space and configured to emit ultraviolet light to the first end, wherein
the second light source unit includes
a second light emitting element that emits ultraviolet light,
a second support portion provided to protrude from an end surface of the second end of the flow path tube toward the first end, provided at a position corresponding to the outlet, and formed to be tapered toward the first end, and
a second accommodation portion that is provided at a front end of the second support portion and accommodates the light emitting element.

9. The fluid sterilization device according to claim 7, wherein
a flow path direction of the outlet coincides with a direction toward a position offset from a central axis of the flow path tube,
the second support portion is provided at a position including the central axis of the flow path tube, and the fluid that circulates around a periphery of the second support portion flows out from the outlet.

10. The fluid sterilization device according to claim 7, wherein
flow path directions of the inlet and the outlet coincide with a direction toward a position offset from a central axis of the flow path tube,
the fluid that flows through the flow path space forms a helical flow, and
the inlet and the outlet are formed such that, when the flow path tube is viewed from a central axis direction, inflow directions of the inlet and the outlet coincide with a tangential direction of rotation of the helical flow centered on the central axis of the flow path tube.

11. The fluid sterilization device according to claim 1, further comprising:
a light intensity sensor that protrudes from an inner wall surface of the flow path tube toward a central axis of the flow path tube.

12. The fluid sterilization device according to claim 1, wherein
the flow path tube includes a helical groove on an inner wall surface of the flow path tube.

13. The fluid sterilization device according to claim 3, wherein
a surface of the accommodation portion at a side of the support portion is provided with a helical groove or a protrusion.

14. The fluid sterilization device according to claim 3, wherein
a surface of the accommodation portion at a side of the support portion is provided with a radial groove or a protrusion.
